(19) United States
(12) Patent Application Publication
(10) Pub. No.: US 2002/0102309 A1
(43) Pub. Date: Aug. 1, 2002

(54) CONTROLLED RELEASE FORMULATION FOR ADMINISTRATION OF AN ANTI-INFLAMMATORY NAPHTHALENE DERIVATIVE

(76) Inventors: JANE C. I. HIRSH, WELLESLEY, MA (US); SHUBHA CHUNGI, SHARON, MA (US)

Correspondence Address: KENYON & KENYON, ONE BROADWAY, NEW YORK, NY, 10004, US

(21) Appl. No.: 09/395,885

(22) Filed: Sep. 14, 1999

(51) Int. Cl. A61K 9/14; A61K 9/16; A61K 9/50

(52) U.S. Cl. 424/489; 424/490

(57) ABSTRACT

An anti-inflammatory pharmaceutical formulation for the oral administration of a nonsteroidal anti-inflammatory drug (NSAID) is provided, wherein the NSAID an anti-inflammatory naphthalene derivative such as nabumetone, 6-methoxy-2-naphthylacetic acid (6-MNA), a fluoronaphthylone, an amido-substituted naphthalene compound, or a nabumetone derivative comprising an acetal, enol acylate or enol ether of nabumetone. The formulation is controlled release, and a preferred formulation is an enterically coated, delayed release dosage form of nabumetone. Methods for using the novel formulation are provided as well; a preferred use is in the treatment of conditions and disorders associated with inflammation.

### TECHNICAL FIELD

This invention relates generally to pharmaceutical formulations for oral administration of a nonsteroidal anti-inflammatory drug (NSAID), and more particularly relates to a controlled release formulation of an anti-inflammatory naphthalene derivative, e.g., a delayed release, enterically coated nabumetone formulation. The invention additionally relates to therapeutic methods wherein the novel controlled release formulation is administered to a patient.

### BACKGROUND

Nonsteroidal anti-inflammatory drugs such as diclofenac, ibuprofen, ketoprofen, naproxen, nabumetone and piroxicam are widely used to relieve mild to moderate pain, to reduce fever, and to treat inflammatory conditions. Nabumetone (4-(6′-methoxy-2′-naphthyl)butan-2-20 one), for example, has been described in U.S. Pat. No. 4,420,639 for use as an anti-inflammatory agent for the treatment of rheumatic and arthritic conditions.

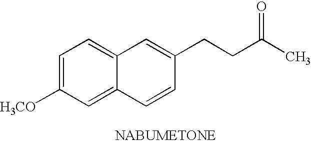

In addition, U.S. Pat. No. 5,695,774 describes the use of nabumetone for the treatment and/or prevention of dementia, such as Alzheimer's disease.

The NSAIDs are non-habit forming drugs and thereby offer a significant advantage over the use of traditional opioid- or steroid-based drugs. However, in some cases, systematic administration of an NSAID is not recommended. NSAIDs can result in systemic toxicity in a host, and since the agent is administered systemically, its effects are also systemic. Thus, the chronic use of NSAIDs or the administration of high doses of NSAIDs has been associated with undesirable side effects such as bleeding, ulceration, and perforation. For example, chronic oral administration of aspirin can result in stomach upset and patient discomfort.

In view of the advantages of NSAIDs, steps have been undertaken to minimize the adverse effects associated with the systemic administration of these drugs. In one approach to reduce the adverse effects of NSAIDs, the agents are ingested with food or milk, or are taken in combination with antacids, histamine H₂-receptor antagonists, omeprazole, or sucraflate. In another approach, NSAIDs have been administered locally, such as by injection or topical administration, or have been co-administered with a prostaglandin.

In yet another approach to reduce the undesirable gastrointestinal effects resulting from systemic administration of NSAIDs, the agents have been enterically coated to give a delayed release formulation. In U.S. Pat. Nos. 3,488,418, 3,341,416, and 3,155,590, aspirin is microencapsulated with ethylcellulose. After ingestion, the gastric fluids slowly diffuse through the encapsulant walls, dissolve the aspirin, and the dissolved aspirin diffuses out through the encapsulant walls into the body. In U.S. Pat. No. 3,906,086, aspirin is coated with a non-aqueous solution of cellulose acetate phthalate. The cellulose acetate phthalate is substantially insoluble in the acidic media of the stomach and the tablet remains intact until it reaches the intestinal tract. Thus, as the cellulose acetate phthalate coating is dissolved by the alkaline intestinal fluid, aspirin is released in the intestinal tract. U.S. Pat. No. 4,966,768 describes a sustained release tablet containing etodolac as the active agent and a mixture of hydroxypropylmethyl cellulose, ethyl cellulose, and dibasic sodium phosphate as a sustained release carrier.

Nabumetone, disclosed in U.S. Pat. No. 4,061,779, is a nonacidic prodrug that is metabolized to an active nonsteroidal antiinflammatory moiety 6-methoxy-2-naphthylacetic acid (6-MNA). 6-MNA is a structural analog of naproxen, and like other NSAIDs, possesses analgesic, antipyretic and anti-inflammatory activity.

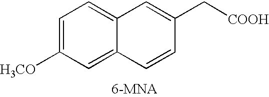

See Pisko et al. (1987), “Nabumetone: a ‘Nonacidic’ Nonsteroidal Antiinflammatory Drug” Pharmatherapeutica 5:90-98. The predominant gastrointestinal reactions associated with NSAIDs, as noted above, include abdominal pain and indigestion, nausea or vomiting; however, the aforementioned publication and others suggest that with nabumetone, duodenal ulcer, gastric ulcer, and gastrointestinal bleeding occurs in less than 1% of patients taking the drug. (See, e.g., S. L. Dahl (1993), “Nabumetone: a ‘Nonacidic’ Nonsteroidal Antiinflammatory Drug” Ann. Pharmacother. 27(4): 456-463, which presents a comparative safety study of patients with osteoarthritis or rheumatoid arthritis using nabumetone, diclofenac, naproxen, piroxicam, and ibuprofen, and concluded that ulcers occurred in 0.03% of nabumetone-treated patients, and in 0.5% of patients using the other NSAIDs.) Thus, the current belief in the art is that nabumetone does not, in general, cause the gastrointestinal reactions usually associated with the administration of NSAIDs, and is therefore safe in relatively high doses and for long-term systemic administration. For this reason, controlled release nabumetone formulations, and particularly delayed release, enterically coated nabumetone compositions, have not been manufactured or made available commercially. However, applicants' own studies have shown that oral administration of an “immediate release” nabumetone formulation results in the side effects associated with other NSAIDs in significantly more patients than previously believed, primarily gastrointestinal reactions such as stomach upset, bleeding, ulceration, and perforation, and have now developed a novel formulation that avoids these side effects.

### SUMMARY OF THE INVENTION

Accordingly, it is a primary object of the invention to address the aforementioned need in the art and provide a controlled release formulation containing an NSAID as the active agent, wherein the NSAID is an anti-inflammatory naphthalene derivative as will be described in detail herein.

It is another object of the invention to provide such a formulation comprising an enterically coated, delayed release dosage form.

It is yet another object of the invention to provide such a formulation wherein the active agent is nabumetone.

It is still another object of the invention to provide such a formulation which significantly reduces the side effects associated with immediate release nabumetone formulations, particularly gastrointestinal reactions such as stomach upset, bleeding, ulceration, and the like.

It is an additional object of the invention to provide a method for treating a patient suffering from a disorder or condition associated with inflammation, comprising orally administering to the patient a dosage form of the invention.

It is a further object of the invention to provide such a method wherein the disorder or condition is a rheumatic or arthritic disease.

In a first embodiment, then, a pharmaceutical formulation is provided comprising a therapeutically effective amount of an anti-inflammatory naphthalene derivative as an active agent, and a polymeric material that, following oral administration, results in a desired “controlled release” profile. The active agent has the structural formula (I)

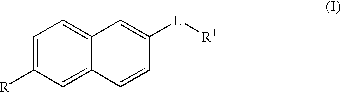

wherein:

R is selected from the group consisting of halo, C₁₋₁₂ alkyl, OR² and SR² wherein R² is C₁₋₁₂ alkyl, C₂₋₆ alkenyl, C₃₋₆ alkynyl, aryl or aralkyl wherein the alkyl group of the aralkyl moiety is C₁₋₃ unsubstituted or substituted with lower alkyl or lower hydroxyalkyl;

L is a linking moiety selected from the group consisting of —CHR³—, —CHR³—CHR⁴—, —CHR³—CO—, —CO—CHR⁴—, —(CO)—, —CHR³—C(OH)R⁴— and —C(R³)═C(R⁴)—, wherein R³ and R⁴ may be the same or different, and are hydrido or lower alkyl; and

R¹ is lower alkyl, —(CH₂)ₙ—COR⁵, —(CH₂)ₙ—COOR⁵, —(CH₂)ₙ—COOH, —(CH₂)ₙ—CH(OH)R⁵, —(CH₂)ₙ—C(CH₃)(OH)R⁵, —(CH₂)ₙ—C(OR⁶)(OR⁷)R⁵, —(CH₂)ₙ—CH═CR⁵(OR⁶), —(CH₂)ₙ—C(OR⁵)═CH₂ or —(CH₂)ₙ—(CO)—N(OR⁸)R⁹, wherein n is 0, 1 or 2, R⁵, R⁶ and R⁷ are independently lower alkyl, R⁸ is hydrido, lower alkyl, phenyl, or lower alkoxy, and R⁹ is hydrido or lower alkyl,

wherein the naphthalene rings may be substituted at one or more available carbon atoms with nonhydrogen substituents such as alkyl, aryl, alkoxy, halo, or the like.

Suitable active agents thus include not only nabumetone per se, i.e., wherein R is methoxy, L is —CH₂—CH₂—, and R¹ is —(CO)CH₃, but other anti-inflammatory naphthalene derivatives such as 6-methoxy-2-naphthylacetic acid, the active metabolite of nabumetone, and fluoronaphthylones (as described in U.S. Pat. No. 4,243,683 to Goudie et al., for example), wherein the naphthalene ring is substituted with a fluorine atom. Other active agents encompassed by structural formula (I) include, but are not limited to, amido-substituted naphthalenes (as described, for example, in U.S. Pat. No. 4,681,894 to Murray et al.), and acetals, enol acylates and enol ethers of nabumetone (as described, for example, in U.S. Pat. No. 4,180,585 to Goudie).

The formulation is preferably an enterically coated, delayed release dosage form in which a core containing the active agent is coated with an enteric polymer effective to delay release of the active agent until the small intestine of the patient is reached. Suitable enteric polymers include, but are not limited to, cellulosic polymers, shellac, zein, and acrylate and/or acrylic acid polymers and copolymers.

In another embodiment of the invention, a method is provided for treating a patient with an NSAID-responsive disorder or condition, typically a disorder or condition associated with inflammation, e.g., a rheumatic or arthritic disease such as osteoarthritis, rheumatoid arthritis, or the like. The method involves orally administering the present controlled release formulation to the patient within the context of a dosage regimen effective to treat the disorder or condition and/or alleviate the inflammation and pain associated therewith.

### DETAILED DESCRIPTION OF THE INVENTION

Overview and Definitions:

Before describing the present invention in detail, it is to be understood that unless otherwise indicated this invention is not limited to specific formulation components, manufacturing methods, dosage regimens, or the like, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, reference to “an active agent” includes a combination of two or more active agents, reference to “an enteric polymer” includes a combination of two or more such polymers, etc.

In describing and claiming the present invention, the following terminology will be used in accordance with the definitions set out below.

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned which also induce the desired pharmacologic effect. The active agents herein are NSAIDs.

The term “controlled release” is intended to refer to any drug-containing formulation in which release of the drug is not immediate, i.e., with a “controlled release” formulation, oral administration does not result in immediate release of the drug into an absorption pool. The term is used interchangeably with “nonimmediate release” as defined in Remington: The Science and Practice of pharmacy, Nineteenth Ed. (Easton, Pa.: Mack Publishing Company, 1995). As discussed therein, immediate and nonimmediate release can be defined kinetically by reference to the following equation:

$$\begin{matrix}Dosage \\ Form\end{matrix} {\begin{matrix}\to \\ \begin{matrix}drug \\ release\end{matrix}\end{matrix}}^{{k}_{r}} \begin{matrix}Absorption \\ Pool\end{matrix} {\begin{matrix}\to \\ absorption\end{matrix}}^{{k}_{a}} \begin{matrix}Target \\ Area\end{matrix} {\begin{matrix}\to \\ elimination\end{matrix}}^{{k}_{e}}$$

The absorption pool represents a solution of the drug administered at a particular absorption site, and kᵣ, kₐ and kₑ are first-order rate constants for (1) release of the drug from the formulation, (2) absorption, and (3) elimination, respectively. For immediate release dosage forms, the rate constant for drug release, kᵣ, is far greater than the absorption rate constant kₐ. For the controlled release formulations, i.e., for the formulations of the present invention, the opposite is true, i.e., kᵣ<<kₐ, such that the rate of release of drug from the dosage form is the rate-limiting step in the delivery of the drug to the target area. The term “controlled release” as used herein is intended to include any nonimmediate release formulation, including but not limited to sustained release, delayed release and pulsatile release formulations.

The term “sustained release” is used in its conventional sense to refer to a drug formulation that provides for gradual release of drug over an extended period of time, and that preferably, although not necessarily, results in substantially constant blood levels of drug over an extended time period.

The term “delayed release” is used in its conventional sense to refer to a drug formulation in which there is a time delay provided between oral administration of a drug dosage form and the release of the drug therefrom. “Delayed release” may or may not involve gradual release of drug over an extended period of time, and thus may or may not be “sustained release.” The preferred “controlled release” formulations herein are “delayed release,” and particularly preferred “delayed release” formulations are enterically coated compositions. “Enteric coating” or “enterically coated” as used herein relates to the presence of polymeric materials in a drug formulation that result in an increase in the drug's resistance to disintegration in the stomach. Typically, the polymeric material is present as a coating surrounding a drug-containing core, but the polymeric material may also be present in admixture with the drug itself within a coated formulation. “Carriers” or “vehicles” as used herein refer to carrier materials suitable for drug administration. Carriers and vehicles useful herein include any such materials known in the art, e.g., any liquid, gel, solvent, liquid diluent, solubilizer, or the like, which is nontoxic and which does not interact with other components of the composition in a deleterious manner.

By the terms “effective amount” or “therapeutically effective amount” of an agent as provided herein are meant a nontoxic but sufficient amount of the agent to provide the desired therapeutic effect. As will be pointed out below, the exact amount required will vary from subject to subject, depending on the species, age, and general condition of the subject, mode of administration, and the like. Thus, it is not possible to specify an exact “effective amount.” However, an appropriate “effective” amount in any individual case may be determined by one of ordinary skill in the art using only routine experimentation.

By “pharmacologically acceptable” is meant a material which is not biologically or otherwise undesirable, i.e., the material may be administered to an individual without causing any undesirable biological effects or interacting in a deleterious manner with any components of the pharmaceutical composition in which it is contained. Thus, a “pharmacologically acceptable” salt or ester of a compound refers to a salt or ester which is not biologically or otherwise undesirable.

The terms “treating” and “treatment” as used herein refer to reduction in severity and/or frequency of symptoms, elimination of symptoms and/or underlying cause, prevention of the occurrence of symptoms and/or their underlying cause, and improvement or remediaton of damage. Thus, for example, the present method of “treating” inflammation, as the term “treating” is used herein, encompasses both prevention of inflammation in a predisposed individual and treatment of inflammation in a clinically symptomatic individual.

The following definitions pertain to chemical structures, molecular segments and substituents:

The term “alkyl” as used herein refers to a branched or unbranched saturated hydrocarbon group of 1 to about 12 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. The term “lower alkyl” intends an alkyl group of 1 to 6 carbon atoms, preferably 1 to 4 carbon atoms.

The term “alkenyl” as used herein refers to a branched or unbranched hydrocarbon group of 2 to about 12 carbon atoms containing at least one double bond, such as ethenyl, n-propenyl, isopropenyl, n-butenyl, isobutenyl, octenyl, decenyl, and the like. Preferred alkenyl groups herein contain 2 to 6 carbon atoms. The term “lower alkenyl” intends an alkenyl group of 2 to 6, preferably 2 to 4 carbon atoms.

The term “alkynyl” as used herein refers to a branched or unbranched hydrocarbon group of 2 to about 12 carbon atoms containing at least one triple bond, such as ethynyl, n-propynyl, isopropynyl, n-butynyl, isobutynyl, octynyl, decynyl, and the like. Preferred alkynyl groups herein contain 3 to 6 carbon atoms. The term “lower alkynyl” intends an alkynyl group of 2 to 6 carbon atoms, preferably 3 or 4 carbon atoms.

The term “alkoxy” as used herein intends an alkyl group bound through a single, terminal ether linkage; that is, an “alkoxy” group may be defined as —O-alkyl where alkyl is as defined above. A “lower alkoxy” group intends an alkoxy group containing one to six, more preferably one to four, carbon atoms.

Similarly, the term “alkyl thio” as used herein intends an alkyl group bound through a single, terminal thioether linkage; that is, an “alkyl thio” group may be defined as -S-alkyl where alkyl is as defined above. A “lower alkyl thio” group intends an alkyl thio group containing 1 to 6, preferably 1 to 4, carbon atoms.

The term “aryl” as used herein, and unless otherwise specified, refers to an aromatic species containing 1 to 3 aromatic rings, either fused or linked, and either unsubstituted or substituted with 1 or more substituents typically selected from the group consisting of lower alkyl, lower alkoxy, halogen, and the like. Preferred aryl substituents contain 1 aromatic ring or 2 fused or linked aromatic rings.

The term “aralkyl” refers to an alkyl group with an aryl substituent, and the term “aralkylene” refers to an alkylene group with an aryl substituent; the term “alkaryl” refers to an aryl group that has an alkyl substituent, and the term “alkarylene” refers to an arylene group with an alkyl substituent.

The terms “halo” and “halogen” are used in the conventional sense to refer to a chloro, bromo, fluoro or iodo substituent. The terms “haloalkyl,” “haloalkenyl” or “haloalkynyl” (or “halogenated alkyl,” “halogenated alkenyl,” or “halogenated alkynyl”) refers to an alkyl, alkenyl or alkynyl group, respectively, in which at least one of the hydrogen atoms in the group has been replaced with a halogen atom.

“Optional” or “optionally” means that the subsequently described circumstance may or may not occur, so that the description includes instances where the circumstance occurs and instances where it does not. For example, the phrase “optionally substituted,” as in a naphthalene derivative “optionally substituted” on one or both of the naphthalene rings, means that a nonhydrogen substituent may or may not be present, and, thus, the description includes structures wherein a nonhydrogen substituent is present and structures wherein a nonhydrogen substituent is not present.

The Controlled Release Formulations:

In a first embodiment, the invention provides a pharmaceutical formulation comprising (1) a therapeutically effective amount of an anti-inflammatory naphthalene derivative as the active agent, and (2) a polymeric material that, following oral administration of the formulation to a patient, is effective to provide a desired controlled release profile, preferably delaying release of the active agent until the small intestine of the patient is reached. Typically, the active agent is present in a core that is coated with a composition containing the polymeric material, i.e., is encapsulated within an “enteric coating,” wherein the polymeric material comprises an “enteric polymer.”

The active agent may be represented generically by the following structural formula (I)

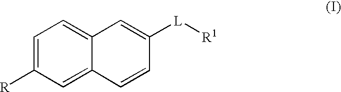

wherein R, L and R¹ are defined as follows:

R is halo, preferably chloro or bromo; C₁₋₁₂ alkyl, preferably lower alkyl; OR²; or SR². R² is: C₁₋₁₂ alkyl, preferably lower alkyl; C₂₋₆ alkenyl; C₃₋₆ alkynyl; aryl, preferably phenyl; or aralkyl wherein the alkyl group is C₁₋₃ unsubstituted or substituted with lower alkyl or lower hydroxyalkyl. Preferred R groups are lower alkoxy and lower alkyl thio, with methoxy and methyl thio more preferred and methoxy most preferred.

L is a linking moiety selected from the group consisting of —CHR³—, —CHR³—CHR⁴—, —CHR³—CO—, —CO—CHR⁴—, —(CO)—, —CHR³—C(OH)R⁴— and —C(R³)═C(R⁴)—, wherein R³ and R⁴ may be the same or different, and are hydrido or lower alkyl. Preferred L moieties are —CHR³—CHR⁴—.

R¹ is lower alkyl, —(CH₂)ₙ—COR⁵, —(CH₂)ₙ—COOR⁵, —(CH₂)ₙ—COOH, —(CH₂)ₙ—CH(OH)R⁵, —(CH₂)ₙ—C(CH₃)(OH)R⁵, —(CH₂)ₙ—C(OR⁶)(OR⁷)R⁵, —(CH₂)ₙ—CH═CR⁵(OR⁶), —(CH₂)ₙ—C(OR⁵)═CH₂ or —(CH₂)ₙ—(CO)—N(OR⁸)R⁹, wherein n is 0, 1 or 2, R⁵, R⁶ and R⁷ are independently lower alkyl, R⁸ is hydrido, lower alkyl, phenyl, or lower alkoxy, and R⁹ is hydrido or lower alkyl. Preferably, R² is —COOH or —COCH₃.

One or more available carbon atoms of the naphthalene rings may be substituted with a non-hydrogen substituent such as alkyl, aryl, alkoxy, halo, or the like.

A particularly preferred active agent within this group is nabumetone, wherein R is methoxy, L is —CH₂—CH₂—, and R¹ is —(CO)CH₃. Other suitable active agents having the structural formula (I) and useful in conjunction with the present invention include 6-methoxy-2-naphthylacetic acid (6-MNA)

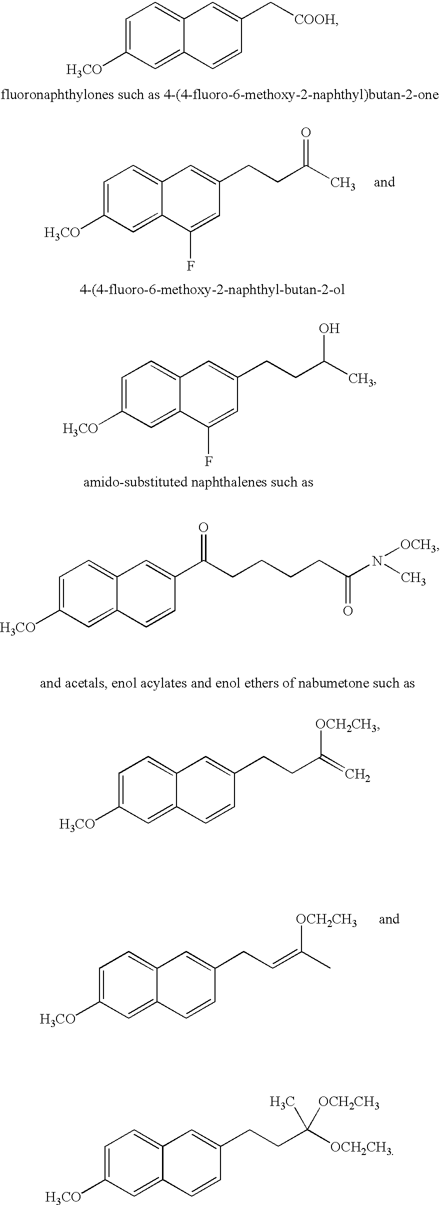

As will be appreciated by those skilled in the art, numerous other anti-inflammatory naphthalene derivatives are encompassed by structural formula (I) and are similarly suitable for incorporation into the present formulations. Such derivatives are described, for example, in U.S. Pat. No. 4,180,585 to Goudie, U.S. Pat. No. 4,243,682 to Goudie et al., and U.S. Pat. No. 4,681,894 to Murray et al. The compounds may be obtained commercially or synthesized using techniques described in the aforementioned patents or in other pertinent texts, patents or literature references.

The therapeutically effective amount of active agent in the formulation is preferably a unit dosage, typically in the range of 250 mg to 1000 mg, preferably in the range of 500 mg to 1000 mg. More preferably, the formulations of the invention are tablets containing 500 mg, 750 mg or 1000 mg active agent, most preferably containing 500 mg or 750 mg active agent. The active agent may be present in the formulation as a salt, ester, amide, or other derivative, or may be functionalized in various ways as will be appreciated by those skilled in the art and as described in the pertinent texts, patents and literature; however, it is preferred that the formulation contain nabumetone per se, i.e., not in derivatized or functionalized form.

In addition to the active agent, the core of the delayed release formulation will generally contain other materials such as binders, diluents, lubricants, disintegrants, fillers, stabilizers, surfactants, coloring agents, and the like. An additional active agent may also be included if desired. Binders are used to impart cohesive qualities to the tablet core, and thus ensure that the core remains intact after compression and prior to coating. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch and powdered sugar. Lubricants are used to facilitate manufacture of the drug-containing core; examples of suitable lubricants include, for example, magnesium stearate, calcium stearate, and stearic acid. Stearates, if present, preferably represent at no more than approximately 2 wt. % of the drug-containing core. Disintegrants are used to facilitate disintegration of the drug-containing core, and are generally starches, clays, celluloses, algins, gums or crosslinked polymers; sodium starch glycolate is particularly preferred. Fillers include, for example, insoluble materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions which include, by way of example, oxidative reactions. Surfactants may be anionic, cationic, amphoteric or nonionic surface active agents, with anionic surfactants preferred. Suitable anionic surfactants include, but are not limited to, those containing carboxylate, sulfonate and sulfate ions, associated with cations such as sodium, potassium and ammonium ions. Particularly preferred surfactants include, but are not limited to: long alkyl chain sulfonates and alkyl aryl sulfonates such as sodium dodecylbenzene sulfonate; dialkyl sodium sulfosuccinates, such as sodium bis-(2-ethylhexyl)-sulfosuccinate; and alkyl sulfates such as sodium lauryl sulfate.

The core of the formulation will generally contain approximately 5 to 95 wt. %, preferably 50 to 95 wt. %, more preferably 60 wt. % to 90 wt. %, and most preferably 75 wt. % to 85 wt. % active agent, with the remainder of the core comprising binders and other materials as described above.

For controlled release compositions other than enteric coated, delayed release dosage forms, the formulations are prepared using conventional components and processing techniques as described, for example, in Remington, supra, and as known to those skilled in the art of pharmaceutical formulation. Such compositions may be diffusion-controlled reservoir or matrix systems, wherein in the former case, a core of drug is surrounded by a water-insoluble polymeric membrane, and in the latter case, the drug is dispersed throughout an inert matrix of a water-insoluble polymeric material.

Preferred formulations herein, however, are enterically coated tablets. The enterically coated tablets of the invention may be manufactured using standard tablet processing procedures and equipment for making the drug-containing core, and conventional coating procedures and equipment for encapsulating the core in an enteric coating. Such procedures are known to those skilled in the art and described in the pertinent texts, e.g., in Remington, supra. A preferred method for forming the drug-containing core is by direct compression of a powdered, crystalline or granular drug-containing composition, alone or in combination with diluents, binders, lubricants, disintegrants, colorants or the like. As an alternative to direct compression, the compressed core can be prepared using wet-granulation or dry-granulation processes. The tablet core may also be molded rather than compressed, starting with a moist material containing a suitable water-soluble lubricant; however, it is preferred that the drug-containing core be manufactured using compression rather than molding. After the drug-containing core is prepared, the enteric coating composition is then applied using a coating pan, an airless spray technique, fluidized bed coating equipment, or the like.

The enteric coating composition comprises a polymeric material that, following oral administration of the formulation to a patient, prevents release of the active agent until the small intestine of the patient is reached. Generally, this requires that the polymeric material—i.e., the enteric polymer—prevent drug release in the acidic environment of the stomach but dissolve sufficiently in the small intestines to gradually release the active agent therein. Suitable enteric polymers include, but are not limited to, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), shellac, zein, and acrylic acid polymers and copolymers, preferably formed from acrylic acid, methacrylic acid, methyl acrylate, ethyl acrylate, methyl methacrylate and/or ethyl methacrylate. Preferred enteric polymers for use herein are copolymers of methacrylic acid and methyl methacrylate. Such copolymers are commercially available under the tradenames Eudragit L and Eudragit S, in which the ratio of free carboxyl to ester groups is approximately 1:1 and 1:2, respectively, and wherein each copolymer has a (weight average) molecular weight of approximately 135,000 Da. Most preferred for use in conjunction with the present invention is the commercially available copolymer known as Eudragit L100-55, which comprises granules of Eudragit L that are soluble at an intestinal pH of 5.5 and above. The amount of enteric coating material used is such that most, i.e., at least about 60%, preferably at least about 75%, of the active agent is released within about two hours of drug administration. In order to achieve the aforementioned release profile, the amount of enteric coating used should be on the order of 5 wt. % to 20 wt. % of the total dosage form, preferably on the order of 5 wt. % to 15 wt. % of the total dosage form.

A preferred enteric coated nabumetone composition of the invention is wherein a drug-containing core comprises:

approximately 60 wt. % to 90 wt. % nabumetone,

approximately 5 wt. % to 15 wt. % sodium starch glycolate,

approximately 0.1 wt. % to 2 wt. % surfactant,

approximately 1 wt. % to 4 wt. % hydroxypropyl methylcellulose,

approximately 2.5 wt. % to 7.5 wt. % polyethylene glycol, and

approximately 0.5 wt. % to 10 wt. % microcrystalline cellulose, with all of the aforementioned percentages representing the fractional portion of the core; and wherein the core is coated with an enteric coating comprised of a polymer of acrylic acid, methacrylic acid, an acrylic acid ester, a methacrylic acid ester, or a combination thereof, wherein the enteric coating represents approximately 5 wt. % to 15 wt. % of the total dosage form.

In a particularly preferred enteric coated composition of the invention, the drug-containing core comprises:

approximately 75 wt. % to 85 wt. % nabumetone,

approximately 8 wt. % to 12 wt. % sodium starch glycolate,

approximately 0.5 wt. % to 1.5 wt. % sodium lauryl sulfate,

approximately 1.5 wt. % to 2.5 wt. % hydroxypropyl methylcellulose,

approximately 5 wt. % to 6 wt. % polyethylene glycol, and

approximately 0.5 wt. % to 1.5 wt. % microcrystalline cellulose, wherein the above percentages represent the fractional portion of the core; and wherein the core is coated with an enteric coating comprised of a copolymer of methacrylic acid and methyl methacrylate, and wherein, as above, the enteric coating represents approximately 5 wt. % to 15 wt. % of the total dosage form.

Treatment of NSAID-Responsive Disorders and Conditions:

The novel formulations are to be administered orally to a mammalian individual within the context of a dosage regimen effective to treat an NSAID-responsive condition or disorder. Typically the formulations are employed as anti-inflammatory and/or analgesic compositions, and may be used to treat individuals suffering from rheumatic or arthritic disorders, including, for example: rheumatoid arthritis (RA), degenerative joint disease (also known as DJD and “osteoarthritis”); juvenile rheumatoid arthritis (JRA); psoriatic arthritis; gouty arthritis; ankylosing spondylitis; and lupus erythematoses such as systemic lupus erythematosus and discoid lupus erythematosus.

Other potential uses for the formulation of the present invention include but are not limited to treating fever (via the anti-pyretic property of NSAIDs) or myocardial infarction (MI), transient ischemic attacks, and acute superficial thrombophlebitis (via inhibition of platelet aggregation). Further non-limiting uses for the present invention include either single or adjuvant therapy for ankylosing spondylitis, bursitis, cancer-related pain, dysmenorrhea, gout, headaches, muscular pain, tendonitis, and pain associated with medical procedures such as dental, gynecological, oral, orthopedic, postpartum and urological proceedures.

The dosage regimen will generally although not necessarily involve drug administration once or twice daily. The amount of active compound administered will, of course, vary from subject to subject and depend on the particular disorder or condition, the severity of the symptoms, the subject's age, weight and general condition, and the judgment of the prescribing physician. Generally, however, a daily dosage of nabumetone using the present formulations will be in the range of approximately 250 mg/day to 1000 mg/day. For other anti-inflammatory naphthalene derivatives encompassed by the structure of formula (I), the daily dosage will be roughly analogous, and an exact dosage regimen for these other anti-inflammatory agents may be readily determined by one of ordinary skill in the art.

It is to be understood that while the invention has been described in conjunction with the preferred specific embodiments thereof, that the description above as well as the examples which follow are intended to illustrate and not limit the scope of the invention. Other aspects, advantages and modifications within the scope of the invention will be apparent to those skilled in the art to which the invention pertains.

All patents, patent applications, journal articles and other reference cited herein are incorporated by reference in their entireties.

Experimental:

The practice of the present invention will employ, unless otherwise indicated, conventional techniques of pharmaceutical formulation and the like, which are within the skill of the art. Such techniques are explained fully in the literature.

In the following example, efforts have been made to ensure accuracy with respect to numbers used (e.g., amounts, temperature, etc.) but some experimental error and deviation should be accounted for. Unless indicated otherwise, temperature is in degrees C and pressure is at or near atmospheric. All reagents were obtained commercially unless otherwise indicated.

### EXAMPLE 1

### Preparation of 500 mg Nabumetone Tablet

An enteric coated nabumetone tablet was made by first preparing the nabumetone-containing core, and then coating the core with an enteric coating. The nabumetone-containing core had the following components:

| Component | Function | Amount in dosage form |
|---|---|---|
| Nabumetone | Active agent | 500 mg |
| Sodium starch glycolate, NF | Disintegrant | 57.0 mg |
| Sodium lauryl sulfate, NF | Surfactant | 5.0 mg |
| Hydroxypropyl methylcellulose, | Binder | 12.0 mg |
| USP, 3 CPS | | |
| Polyethylene glycol 8000, | Lubricant | 32.7 mg |
| powdered, NF | | |
| Microcrystalline cellulose | Filler | 6.7 mg |
| (Avicel ®) PH 101, NF | | |

The drug-containing core was prepared using a wet granulation technique, as follows. A granulating solution was first prepared by mixing the sodium lauryl sulfate, hydroxypropyl methylcellulose and water, ensuring thorough wetting of the solid particles and an even dispersion. Separately, the nabumetone and approximately 70 wt. % of the total sodium starch glycolate were mixed and thoroughly blended. The granulating solution was then sprayed into the blend and granulation was conducted for about 15 minutes. The mixture was then dried and screened. Then, the remaining sodium starch glycolate, the polyethylene glycol and the microcrystalline cellulose were added to the dried nabumetone admixture, and blending was conducted for about 20 minutes. The final blend was compressed using a tablet press.

An enteric coating solution was then prepared by slowly adding 1200.0 g Eudragit L100-55 to 2400.0 g water (USP) and mixing for approximately 10 to 15 minutes to ensure homogeneous wetting of the polymer powder. Then, 400.0 g 1N sodium hydroxide was added, and mixing was continued for approximately 30 to 45 minutes. The mixture was then filtered through a No. 40 mesh stainless steel screen. The nabumetone cores (12 kg) were then coated with the solution using a conventional (Accelacota™) coating pan.

### EXAMPLE 2

### Preparation of 750 mg Nabumetone Tablet

The procedure of Example 1 was then used to prepare enteric-coated nabumetone tablets containing 750 mg active agent, wherein the drug-containing core had the following components:

| Component | Function | Amount in dosage form |
|---|---|---|
| Nabumetone | Active agent | 750 mg |
| Sodium starch glycolate, NF | Disintegrant | 85.5 mg |
| Sodium lauryl sulfate, NF | Surfactant | 7.5 mg |
| Hydroxypropyl methylcellulose, | Binder | 18.0 mg |
| USP, 3 CPS | | |
| Polyethylene glycol 8000, | Lubricant | 49.0 mg |
| powdered, NF | | |
| Microcrystalline cellulose | Filler | 10.0 mg |
| (Avicel ®) PH 101, NF | | |

Preparation of the enteric coating solution was carried out as described in Example 1, using 1200.0 g Eudragit L100-55, 2400.0 g water (USP), and 400.0 g 1N sodium hydroxide. The nabumetone cores (12 kg), as in Example 1, were coated with the solution using a conventional coating pan.

### EXAMPLE 3

### Tablet Evaluation—Dissolution and Disintegration Studies

(a) Disintegration Studies:

The enteric efficiency of the enteric coated nabumetone tablets prepared in Examples 1 and 2 was evaluated using a modified USP test. Six of each strength of the enteric coated nabumetone tablets were examined in a modified disintegration tester for 1 hour, using simulated gastric fluid (“SGF”; pH 1.2) as the test medium at 37° C. Subsequently the disintegration time in simulated intestinal fluid (“SIF”) was also evaluated. Results indicated an ability of both strengths to resist breakdown for the prescribed period, with a disintegration time in the SIF solution of approximately 15 minutes, and a corresponding disintegration time of approximately 10 minutes for the uncoated cores.

(b) Dissolution Studies:

An evaluation of the release of each strength of the prepared nabumetone tablets in SGF over a two-hour period was carried out using a USP dissolution apparatus (paddle method-75 rpm). After two hours, no nabumetone release, through the coatings, was detected. Dissolution was continued in 0.75% sodium lauryl sulfate in DI water and a comparison was made with uncoated tablets.

Results are summarized in the following table:

| | | % Nabumetone Released |
|---|---|---|
| Time | Tablet Type | Mean ± S.D. |
| 15 min. | Enteric | 1.0 ± 0.0 |
| | Uncoated | 73 ± 1.5 |
| 30 min. | Enteric | 8.0 ± 5.6 |
| | Uncoated | 83 ± 2.9 |
| 60 min. | Enteric | 64 ± 8.8 |
| | Uncoated | 89 ± 0.2 |
| 120 min. | Enteric | 81 ± 4.7 |
| | Uncoated | 101 ± 0.4 |
| 180 min. | Enteric | 94 ± 3.9 |
| | Uncoated | 102 ± 3.8 |

### EXAMPLE 4

### Comparative Testing with Uncoated, Immediate Release Tablets

The compositions prepared may be compared with immediate release nabumetone formulations with regard to therapeutic efficacy and gastrointestinal side effects, and will be found to be far superior, insofar as the compositions of the invention are equally effective in treating NSAID-responsive conditions and disorders, while resulting in significantly reduced gastrointestinal side effects.

At least 50 patients with rheumatoid arthritis or osteoarthritis are given 1500 mg nabumetone daily using 500 mg or 750 mg immediate release tablets, and, for purposes of comparison, at least 50 patients with rheumatoid arthritis or osteoarthritis are given 1500 mg nabumetone daily using 500 mg or 750 mg enteric coated tablets as prepared in Example 1 or 2, for chronic treatment of the arthritic symptoms. After six weeks of treatment both groups are expected to report improved pain and inflammation management. The group receiving the enteric coated tablets, however, are expected to report statistically significant reduction in gastrointestinal complaints.

1. A pharmaceutical formulation comprising: (a) a therapeutically effective amount of an anti-inflammatory naphthalene derivative as an active agent; and (b) a polymeric material that, upon oral administration of the formulation to a patient, provides for controlled release of the active agent.

2. The formulation of claim 1, wherein the active agent has the structural formula (I) 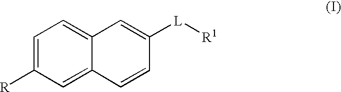
wherein:
R is selected from the group consisting of halo, C₁₋₁₂ alkyl, OR² and SR² wherein R² is C₁₋₁₂ alkyl, C₂₋₆ alkenyl, C₃₋₆ alkynyl, C₁₋₁₂ alkoxy, C₁₋₁₂ alkyl thio, aryl or aralkyl wherein the alkyl group is C₁₋₃ unsubstituted or substituted with lower alkyl or lower hydroxyalkyl;
L is a linking moiety selected from the group consisting of —CHR³—, —CHR³—CHR⁴—, —CHR³—CO—, —CO—CHR⁴—, —(CO)—, —CHR³—C(OH)R⁴— and —C(R³)═C(R⁴)—, wherein R³ and R⁴ may be the same or different, and are hydrido or lower alkyl; and
R¹ is lower alkyl, —(CH₂)ₙ—COR⁵, —(CH₂)ₙ—COOR⁵, —(CH₂)ₙ—COOH, —(CH₂)ₙ—CH(OH)R⁵, —(CH₂)ₙ—C(CH₃)(OH)R⁵, —(CH₂)ₙ—C(OR⁶)(OR⁷)R⁵, —(CH₂)ₙ—CH═CR⁵(OR⁶), —(CH₂)ₙ—C(OR⁵)═CH₂ or —(CH₂)ₙ—(CO)—N(OR⁸)R⁹, wherein n is 0, 1 or 2, R⁵, R⁶ and R⁷ are independently lower alkyl, R⁸ is hydrido, lower alkyl, phenyl, or lower alkoxy, and R⁹ is hydrido or lower alkyl,
wherein the naphthalene rings may be substituted at one or more available carbon atoms with nonhydrogen substituents selected from the group consisting of alkyl, aryl, alkoxy, and halo.

3. The formulation of claim 2, wherein R is lower alkoxy.

4. The formulation of claim 3, wherein the active agent is nabumetone.

5. The formulation of claim 3, wherein the active agent is 6-methoxy-2-naphthylacetic acid.

6. The formulation of claim 2, wherein the active agent is a fluoronaphthylone.

7. The formulation of claim 6, wherein the fluoronaphthylone is selected from the group consisting of 4-(4-fluoro-6-methoxy-2-naphthyl)butan-2-one and 4-(4-fluoro-6-methoxy-2-naphthyl)butan-2-ol.

8. The formulation of claim 3, wherein the active agent is an acetal, enol acylate or enol ether of nabumetone.

9. A delayed release, enteric coated nabumetone formulation, comprising an inner core of a nonsteroidal anti-inflammatory composition containing an active agent selected from the group consisting of nabumetone, 6-methoxy-2-naphthylacetic acid, fluoronaphthylones, and acetals, enol acylates and enol ethers of nabumetone, coated with an enteric coating composition comprising an enteric polymer that, upon oral administration of the formulation to a patient, prevents release of the active agent until the small intestine of the patient is reached.

10. The formulation of claim 9, wherein the active agent is nabumetone or 6-methoxy-2-naphthylacetic acid.

11. The formulation of claim 10, wherein the active agent is nabumetone.

12. The formulation of claim 10, wherein the active agent is 6-methoxy-2-naphthylacetic acid.

13. The formulation of claim 11, in unit dosage form.

14. The formulation of claim 12, in unit dosage form.

15. The formulation of claim 13, comprising approximately 250 mg to 1000 mg nabumetone.

16. The formulation of claim 15, comprising approximately 500 mg to 750 mg nabumetone.

17. The formulation of claim 9, comprising approximately 5 to 95 wt. % active agent.

18. The formulation of claim 11, comprising approximately 5 to 95 wt. % active agent.

19. The formulation of claim 12, comprising approximately 5 to 95 wt. % active agent.

20. The formulation of claim 9, wherein the inner core further comprises at least one additional component selected from the group consisting of diluents, lubricants, disintegrants, fillers, stabilizers and coloring agents.

21. The formulation of claim 9, wherein the enteric polymer is selected from the group consisting of cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), shellac, zein, and polymers and copolymers of acrylic acid and acrylic acid esters.

22. The formulation of claim 21, wherein the enteric polymer is an acrylic acid polymer or copolymer.

23. The formulation of claim 22, wherein the comprises a polymer or copolymer of methyl acrylate, ethyl acrylate, methyl methacrylate, ethyl methacrylate, acrylic acid, and/or methacrylic acid.

24. A method for treating inflammation in a patient, comprising orally administering to the patient the pharmaceutical formulation of claim 1.

25. A method for treating inflammation in a patient, comprising orally administering to the patient the pharmaceutical formulation of claim 9.

26. A method for treating inflammation in a patient, comprising orally administering to the patient a pharmaceutical formulation comprising an enteric coated nabumetone composition, wherein the nabumetone composition is in the form of a compressed core comprising 5 to 95 wt. % nabumetone, coated with an enteric coating composition comprising an enteric polymer selected from the group consisting of cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, and polymers and copolymers of acrylic acid and acrylic acid esters.

27. A method for treating a patient afflicted with an NSAID-responsive condition or disorder, comprising orally administering to the patient the pharmaceutical formulation of claim 1.

28. A method for treating a patient afflicted with an NSAID-responsive condition or disorder, comprising orally administering to the patient the pharmaceutical formulation of claim 9.

29. A method for treating a patient afflicted with an NSAID-responsive condition or disorder, comprising orally administering to the patient a pharmaceutical formulation comprising an enteric coated nabumetone composition, wherein the nabumetone composition is in the form of a compressed core comprising 5 to 95 wt. % nabumetone, coated with an enteric coating composition comprising an enteric polymer selected from the group consisting of cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, and acrylic acid polymers and copolymers.

30. The method of claim 27, wherein the NSAID-responsive condition or disorder is a rheumatic or arthritic disease.

31. The method of claim 30, wherein the disease is rheumatoid arthritis.

32. The method of claim 30, wherein the disease is osteoarthritis.

33. The method of claim 28, wherein the NSAID-responsive condition or disorder is a rheumatic or arthritic disease.

34. The method of claim 33, wherein the disease is rheumatoid arthritis.

35. The method of claim 33, wherein the disease is osteoarthritis.

34. The method of claim 29, wherein the NSAID-responsive condition or disorder is a rheumatic or arthritic disease.

35. The method of claim 34, wherein the disease is rheumatoid arthritis.

36. The method of claim 34, wherein the disease is osteoarthritis.

37. A method for reducing the side effects associated with the oral administration of nabumetone, comprising administering the nabumetone in an enteric coated nabumetone composition, wherein the nabumetone composition is a compressed core comprising 5 to 95 wt. % nabumetone, coated with an enteric coating composition comprising an enteric polymer selected from the group consisting of cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, and acrylic acid polymers and copolymers.
